(19) United States
(12) Patent Application Publication
(10) Pub. No.: US 2004/0097740 A1
(43) Pub. Date: May 20, 2004

(54) Method for producing delta 1-pyrrolines

(76) Inventors: Andrew Plant, Bershire (GB); Fritz Maurer, Monheim (DE)

Correspondence Address: BAYER CROPSCIENCE LP, Patent Department, 100 BAYER ROAD, PITTSBURGH, PA, 15205-9741, US

(21) Appl. No.: 10/380,787

(22) PCT Filed: Sep. 10, 2001
(86) PCT No.: PCT/EP01/10431

(30) Foreign Application Priority Data
Sep. 22, 2000 (DE) 100 47 111.0

(51) Int. Cl. C 07D 2 7/10

(52) U.S. Cl. 548/577

(57) ABSTRACT

2,5-Bisaryl-Δ¹-pyrrolines of the formula (I)

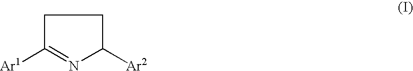

can be prepared by reacting

azides of the formula (II)

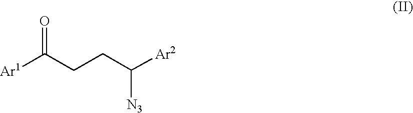

with a trialkylphosphine or a triarylphoshine or a trialkyl phosphite or a reducing agent in the presence of a diluent and, if appropriate, in the presence of a catalyst, Ar¹ and Ar² being as defined in the description.

The present invention relates to a novel process for preparing 2,5-bisaryl-Δ¹-pyrrolines.

Δ¹-Pyrrolines, processes for their preparation and their use as pesticides have already been described in WO 00/21958, WO 99/59968, WO 99/59967 and WO 98/22438. However, these processes are unsatisfactory with respect to the yields, the practice of the reaction, the number of by-products, the type of work-up, the amount of waste produced and the energy consumption. Accordingly, there is a constant need for novel processes which overcome one or more of the disadvantages mentioned.

It has now been found that 2,5-bisaryl-Δ¹-pyrrolines of the formula (I)

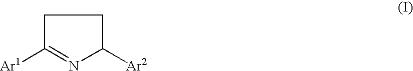

in which

Ar¹ represents the radical

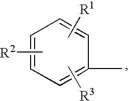

Ar² represents the radical

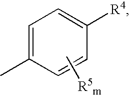

m represents 0, 1, 2, 3 or 4

R¹ represents halogen, cyano, nitro, alkyl, alkoxy, haloalkyl, haloalkoxy, alkoxyalkyl, —S(O)ₒR⁶ or —NR⁷R⁸,

R² and R³ independently of one another represent hydrogen, halogen, cyano, nitro, alkyl, alkoxy, haloalkyl, haloalkoxy, alkoxyalkyl, —S(O)ₒR⁶ or —NR⁷R⁸,

R⁴ represents halogen or one of the groupings below

(l) —X—A

(m) —B—Z-D

(n) —Y-E,

R⁵ represents halogen, hydroxyl, alkyl, alkoxy, haloalkyl, haloalkoxy, trialkylsilyl, alkoxycarbonyl, —CONR⁷R⁸, —S(O)ₒR⁶ or —NR⁷R⁸,

X represents a direct bond, oxygen, —S(O)ₒ—, NR₆—, carbonyl, carbonyloxy, oxycarbonyl, oxysulphonyl (OSO₂), alkylene, alkenylene, alkynylene, alkylenoxy, oxyalkylene, oxyalkylenoxy, —S(O)ₒ-alkylene, cyclopropylene or oxiranylene,

A represents phenyl, naphthyl or tetrahydronaphthyl, each of which is optionally mono- or polysubstituted by radicals from the list W¹, or represents 5- to 10-membered saturated or unsaturated heterocyclyl which contains one or more heteroatoms from the group consisting of nitrogen, oxygen and sulphur and is in each case optionally mono- or polysubstituted by radicals from the list W²,

B represents p-phenylene which is optionally mono- or disubstituted by radicals from the list W¹,

Z represents —(CH₂)ₙ—, oxygen or —S(O)ₒ—,

D represents hydrogen, alkyl, alkenyl, alkynyl, haloalkyl, haloalkenyl, haloalkylsulphonyl or dialkylaminosulphonyl,

Y represents a direct bond, oxygen, sulphur, —SO₂—, carbonyl, carbonyloxy, oxycarbonyl, alkylene, alkenylene, alkynylene, haloalkylene, haloalkenylene, alkylenoxy, oxyalkylene, oxyalkylenoxy or thioalkylene,

E represents hydrogen, alkyl, alkenyl, alkynyl, haloalkyl, haloalkenyl, haloalkylsulphonyl or dialkylaminosulphonyl,

W¹ represents cyano, halogen, formyl, nitro, alkyl, trialkylsilyl, alkoxy, haloalkyl, haloalkenyl, haloalkoxy, haloalkenyloxy, alkylcarbonyl, alkoxycarbonyl, —S(O)ₒR⁶ or —SO₂NR⁷R⁸,

W² represents cyano, halogen, formyl, nitro, alkyl, trialkylsilyl, alkoxy, haloalkyl, haloalkoxy, haloalkenyloxy, alkylcarbonyl, alkoxycarbonyl or —S(O)ₒR⁶,

n represents 0, 1, 2, 3 or 4,

o represents 0, 1 or 2,

R⁶ represents hydrogen, alkyl or haloalkyl,

R⁷ and R⁸ independently of one another represent hydrogen, alkyl, haloalkyl, or together represent alkylene or alkoxyalkylene,

can be prepared by reacting

azides of the formula (II)

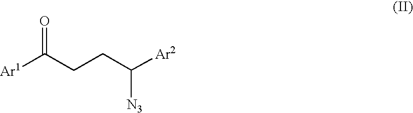

in which Ar¹ and Ar² are as defined above

with a trialkylphosphine or a triarylphoshine or a trialkyl phosphite or a reducing agent in the presence of a diluent and, if appropriate, in the presence of a catalyst.

It is extremely surprising that 2,5-bisaryl-Δ¹-pyrrolines of the formula (I) can be prepared by the process according to the invention in a smooth reaction without interfering side reactions.

The process according to the invention has a number of advantages. Thus, the process according to the invention is clearly superior to the processes known from the prior art since it allows a wider range of starting materials to be used (cf. WO 98/22438). Moreover, in the processes according to the invention, no regioisomers are formed, giving the products of the formula (I) in a higher yield. A further advantage compared to the prior art is that fact that the processes according to the invention dispense with the use of organometallic compounds, which allows an industrially more favourable access to the target compounds. Moreover, the use of the process according to the invention offers the advantage that the energy requirements for the practice can be reduced since many reaction steps proceed at from 0° C. to 40° C., frequently even with particular preference at room temperature.

Using 4-(4-bromophenyl)-4-azido-1-(2,6-difluorophenyl)-1-butanone and triphenylphosphine as starting materials, the course of the process according to the invention can be illustrated by the formula scheme below.

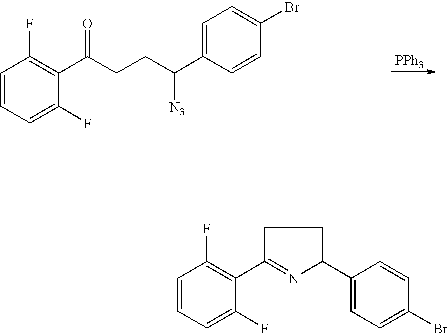

The formula (II) provides a general definition of the azides required as starting materials for carrying out the process according to the invention.

Preferred substituents or ranges of the radicals in the formulae of starting materials of the formula (II) mentioned above and below are illustrated below.

Ar¹ preferably represents the radical

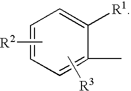

Ar² preferably represents the radical

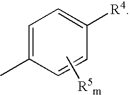

m preferably represents 0, 1, 2 or 3.

R¹ preferably represents halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)_{O}R⁶ or —NR⁷R⁸.

R² and R³ independently of one another preferably represent hydrogen, halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₙR⁶ or —NR⁷R⁸.

R⁴ preferably represents fluorine, chlorine, bromine, iodine or one of the groupings below

(l) —X-A

(m) —B—Z-D

(n) —Y-E.

R⁵ preferably represents halogen, hydroxyl, C₁-C₆-alkyl, C₁-C₆-alkoxy, C₁-C₆-haloalkyl, C₁-C₆-haloalkoxy, tri(C₁-C₆-alkyl)silyl, C₁-C₆-alkoxycarbonyl, —CONR⁷R⁸, —S(O)ₙR⁶ or —NR⁷R⁸.

X preferably represents a direct bond, oxygen, —S(O)ₒ—, —NR⁶—, carbonyl, carbonyloxy, oxycarbonyl, oxysulphonyl (OSO₂), C₁-C₄-alkylene, C₂-C₄alkenylene, C₂-C₄-alkynylene, C₁-C₄-alkylenoxy, C₁-C₄-oxyalkylene, C₁-C₄-oxyalkylenoxy, —S(O)ₒ—C₁-C₄-alkylene, cyclopropylene or oxiranylene.

A preferably represents phenyl, naphthyl or tetrahydronaphthyl, each of which is optionally mono- to tetrasubstituted by radicals from the list W¹, or represents 5- to 10-membered heterocyclyl which contains 1 or 2 aromatic rings and 1 to 4 heteroatoms, selected from a combination of 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl), and is in each case optionally mono- to tetrasubstituted by radicals from the list W².

B preferably represents p-phenylene which is optionally mono- or disubstituted by radicals from the list W¹.

Z preferably represents —(CH₂)ₙ—, oxygen or —S(O)ₒ—.

D preferably represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆alkyl)aminosulphonyl.

Y preferably represents a direct bond, oxygen, sulphur, —SO₂—, carbonyl, carbonyloxy, oxycarbonyl, C₁-C₆-alkylene, C₂-C₆-alkenylene, C₂-C₆alkynylene, C₁-C₆-haloalkylene, C₂-C₆-haloalkenylene, C₁-C₄-alkylenoxy, C₁-C₄-oxyalkylene, C₁-C₄-oxyalkylenoxy or C₁-C₄-thioalkylene.

E preferably represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆alkyl) amino sulphonyl.

W¹ preferably represents cyano, halogen, formyl, nitro, C₁-C₆-alkyl, tri(C₁-C₄alkyl)silyl, C₁-C₆-alkoxy, C₁-C₆-haloalkyl, C₂-C₆-haloalkenyl, C₁-C₆-haloalkoxy, C₂-C₆-haloalkenyloxy, C₁-C₆-alkylcarbonyl, C₁-C₆alkoxycarbonyl, —S(O)ₒR⁶ or —SO₂NR⁷R⁸.

W² preferably represents cyano, halogen, formyl, nitro, C₁-C₆-alkyl, tri(C₁-C₄alkyl)silyl, C₁-C₆-alkoxy, C₁-C₆-haloalkyl, C₁-C₆-haloalkoxy, C₂-C₆-haloalkenyloxy, C₁-C₆-alkylcarbonyl, C₁-C₆-alkoxycarbonyl or —S(O)ₒR⁶.

n preferably represents 0, 1, 2, 3 or 4.

o preferably represents 0, 1 or 2.

R⁶ preferably represents hydrogen, C₁-C₆-alkyl or C₁-C₆-haloalkyl.

R⁷ and R⁸ independently of one another preferably represents hydrogen, C₁-C₆-alkyl, C₁-C₆-haloalkyl, or together represent C₂-C₆-alkylene or C₁-C₄-alkoxy-C₁-C₄-alkylene (for example morpholine).

Ar¹ particularly preferably represents the radical

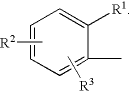

Ar² particularly preferably represents the radical

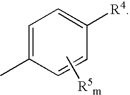

m particularly preferably represents 0, 1 or 2.

R¹ particularly preferably represents fluorine, chlorine, bromine, C₁-C₆-alkyl, C₁-C₆-alkoxy, in each case fluorine- or chlorine-substituted C₁-C₆-alkyl or C₁-C₆-alkoxy.

R² and R³ independently of one another particularly preferably represent hydrogen, fluorine, chlorine, bromine, iodine, C₁-C₆-alkyl, C₁-C₆-alkoxy, in each case fluorine- or chlorine-substituted C₁-C₆-alkyl or C₁-C₆-alkoxy.

R⁴ particularly preferably represents chlorine, bromine, iodine or one of the groupings below

(l) —X-A

(m) —B—Z-D

(n) —Y-E.

R⁵ particularly preferably represents fluorine, chlorine, bromine, iodine, hydroxyl, C₁-C₆-alkyl, C₁-C₆-alkoxy, in each case fluorine- or chlorine-substituted C₁-C₆-alkyl or C₁-C₆-alkoxy, C₁-C₄-alkoxycarbonyl, —CONR⁷R⁸, —S(O)ₒR⁶ or —NR⁷R⁸.

X particularly preferably represents a direct bond, oxygen, sulphur, —SO₂—, carbonyl, carbonyloxy, oxycarbonyl, oxysulphonyl (OSO₂), C₁-C₄-alkylene, C₂-C₄-alkenylene, C₂-C₄-alkynylene, C₁-C₄-alkylenoxy, C₁-C₄-oxyalylene, C₁-C₄-oxyalkylenoxy, —S(O)ₒ—C₁-C₄-alkylene, cyclopropylene or oxiranylene.

A particularly preferably represents phenyl, naphthyl or tetrahydronaphthyl, each of which is optionally mono- to trisubstituted by radicals from the list W¹, or represents 5- to 10-membered heterocyclyl which contains 1 or 2 aromatic rings and 1 to 4 heteroatoms, selected from a combination of 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl), and is in each case optionally mono- to trisubstituted by radicals from the list W².

B particularly preferably represents p-phenylene which is optionally mono- or disubstituted by radicals from the list W¹.

Z particularly preferably represents —(CH₂)ₙ—, oxygen or —S(O)ₒ—.

D particularly preferably represents hydrogen, C₁-C₆-alkyl, C₂-C₆-alkenyl, C₂C₆-alkynyl; in each case fluorine- or chlorine-substituted C₁-C₆-alkyl, C₂-C₆alkenyl or C₁-C₄-alkylsulphonyl; or represents di(C₁-C₄alkyl)aminosulphonyl.

Y particularly preferably represents a direct bond, oxygen, sulphur, —SO₂—, carbonyl, carbonyloxy, oxycarbonyl, C₁-C₆-alkylene, C₂-C₆-alkenylene, C₂C₆-alkynylene; in each case fluorine- or chlorine-substituted C₁-C₆-alkylene or C₂-C₆-alkenylene; represents C₁-C₄-alkylenoxy, C₁-C₄-oxyalkylene, C₁-C₄-oxyalkylenoxy or C₁-C₄-thioalkylene.

E particularly preferably represents hydrogen, C₁-C₆-alkyl, C₂-C₆-alkenyl, C₂-C₆-alkynyl; in each case fluorine- or chlorine-substituted C₁-C₆-alkyl, C₂-C₆alkenyl or C₁-C₆-alkylsulphonyl; or represents di(C₁-C₆alkyl)aminosulphonyl.

W¹ particularly preferably represents cyano, fluorine, chlorine, bromine, iodine, formyl, nitro, C₁-C₄-alkyl, C₁-C₄-alkoxy; in each case fluorine- or chlorine-substituted C₁-C₄-alkyl, C₂-C₄-alkenyl, C₁-C₄-alkoxy or C₂-C₆-alkenyloxy; or represents C₁-C₄-alkylcarbonyl, C₁-C₄-alkoxycarbonyl, —S(O)ₒR⁶ or —SO₂NR⁷R⁸. W² particularly preferably represents cyano, fluorine, chlorine, bromine, formyl, nitro, C₁-C₄-alkyl, C₁-C₄-alkoxy; in each case fluorine- or chlorine-substituted C₁-C₄-alkyl, C₁-C₄-alkoxy or C₂-C₆-alkenyloxy; or represents C₁-C₄-alkylcarbonyl, C₁-C₄-alkoxycarbonyl, —S(O)ₒR⁶.

n particularly preferably represents 0, 1, 2 or 3.

o particularly preferably represents 0, 1 or 2.

R⁶ particularly preferably represents C₁-C₆-alkyl or in each case fluorine- or chlorine-substituted methyl or ethyl.

R⁷ and R⁸ independently of one another particularly preferably represent C₁-C₆alkyl, in each case fluorine- or chlorine-substituted C₁-C₆-alkyl, or together represent C₄-C₅-alkylene or represent —(CH₂)₂—O—(CH₂)₂—.

Ar¹ very particularly preferably represents the radical

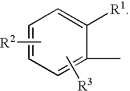

Ar² very particularly preferably represents the radical

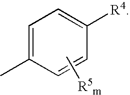

m very particularly preferably represents 0, 1 or 2.

R¹ very particularly preferably represents fluorine, chlorine, bromine, methyl or methoxy.

R² and R³ independently of one another very particularly preferably represent hydrogen, fluorine, chlorine, bromine, methyl or methoxy.

R⁴ very particularly preferably represents chlorine, bromine or one of the groupings below

(l) —X-A

(m) —B—Z-D

(n) —Y-E.

R⁵ very particularly preferably represents fluorine, chlorine, bromine, hydroxyl, methyl, ethyl, methoxy, ethoxy, trifluoromethyl, difluoromethoxy, trifluoromethoxy, —CO₂CH₃ or —SO₂CF₃.

X very particularly preferably represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S(O)O—CH₂— or —S(O)O—(CH₂)₂—.

A very particularly preferably represents phenyl which is optionally mono- or disubstituted by radicals from the list W¹, or represents tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, each of which is optionally mono- or disubstituted by radicals from the list W².

B very particularly preferably represents p-phenylene which is optionally monosubstituted by radicals from the list W¹.

Z very particularly preferably represents oxygen, sulphur or —SO₂—.

D ver particularly preferably represents hydrogen, methyl, ethyl, n-propyl, isopropyl, n-butyl, isobutyl, sec-butyl, tert-butyl, 2-propenyl, butenyl, propargyl, butynyl, —CF₃, —CBF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂.

Y very particularly preferably represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S—CH₂— or —S(CH₂)₂—.

E very particularly preferably represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂.

W¹ very particularly preferably represents cyano, fluorine, chlorine, bromine, formyl, methyl, n-butyl, isobutyl, sec-butyl, tert-butyl, methoxy, ethoxy, n-propoxy, n-butoxy, isobutoxy, sec-butoxy, tert-butoxy, trifluoromethoxy, difluoromethoxy, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —OCH₂CF₃, —SCF₃, —SCHF₂, —SOCHF₂, —SO₂CHF₂, —SOCF₃, —SO₂CF₃ or —SO₂NMe₂W² very particularly preferably represents fluorine, chlorine, bromine, methyl, isopropoxy, tert-butoxy, trifluoromethyl, trifluoromethoxy, difluoromethoxy, trifluoromethylthio, —CO₂CH₃ or —SO₂CF₃.

o very particularly preferably represents 0, 1 or 2.

Particularly preferred starting materials for the process according to the invention are the compounds of the formulae

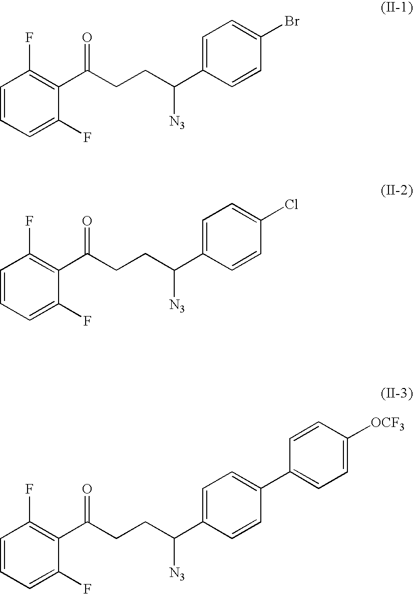

In the definitions mentioned above, oxyalkylene and thioalkylene represent —O-alkyl- and —S-alkyl-, respectively, where the attachment, for example to Ar², is via the oxygen and sulphur atom, respectively, and further substituents may be attached to the alkyl radical, such as, for example, A in —X-A. Alkylenoxy and alkylenethio represent -alkyl-O— and -alkyl-S—, respectively, where the attachment, for example to Ar², is in each case via the alkyl radical and, if appropriate, further substituents may be attached to the oxygen and sulphur atom, respectively, such as, for example, A in —X-A. Oxyalkylenoxy represents —O-alkyl-O.

In the present description, heterocyclyl represents a cyclic hydrocarbon in which one or more carbons are replaced by one or more heteroatoms. Preferred heteroatoms are O, S, N, P, in particular O, S and N.

Preferred, particularly preferred and very particularly preferred are compounds carrying the substituents mentioned under preferred, particularly preferred and very particularly preferred, respectively.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals may be mono- or polysubstituted, where in the case of polysubstitution the substituents may be identical or different. A plurality of radicals having the same indices, such as, for example, m radicals R⁵ for m>1, can be identical or different.

Halogen-substituted radicals, such as, for example, haloalkyl, are mono-or polyhalogenated. In the case of polyhalogenation, the halogen atoms can be identical or different. Here, halogen represents fluorine, chlorine, bromine or iodine, in particular fluorine or chlorine.

However, the abovementioned general or preferred radical definitions or illustrations can also be combined with one another as desired, i.e. between the respective ranges and preferred ranges. The definitions apply both to the end products and, correspondingly, to precursors and intermediates.

Some of the azides of the formula (II) required as starting materials for carrying out the process according to the invention are known. Azides of the formula (II-a)

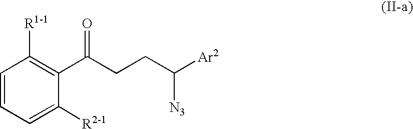

in which

R¹⁻¹ represents fluorine or chlorine,

R²⁻¹ represents hydrogen, fluorine or chlorine and

Ar² is as defined above

are novel.

Azides of the formula (II-a) can be prepared by reacting

a) halides of the formula (III-a)

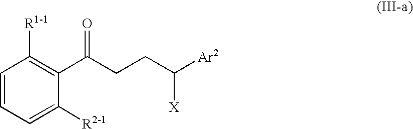

in which R¹⁻¹, R²⁻¹ and Ar² are as defined above and

X represents halogen,

with azides of the formula (IV)

Q-N₃ (IV)

in which

Q represents a cation

in the presence of a diluent and, if appropriate, in the presence of a catalyst.

Azides of the formula (E) can be prepared analogously. To this end, halides of the formula (III)

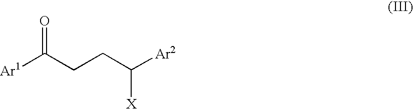

in which Ar¹, Ar² and X are as defined above

are reacted according to process (a).

The formula (III-a) provides a general definition of the halides required as starting materials for carrying out the process (a) according to the invention. In this formula, Ar² preferably, particularly preferably and very particularly preferably has those meanings which have already been mentioned in connection with the description of the starting materials of the formula (II) as being preferred, particularly preferred and very particularly preferred, respectively, for these radicals. R¹⁻¹ preferably represents fluorine or chlorine, R²⁻¹ preferably represents hydrogen, fluorine or chlorine. X preferably represents chlorine, bromine or iodine, particularly preferably chlorine or bromine, very particularly preferably chlorine.

The formula (IV) provides a general definition of the azides required as starting materials for carrying out the process (a) according to the invention. In this formula, Q preferably represents alkali metal ions, trialkylsilyl, tetraalkylammonium, tetralkylguanidinium or polymer-bound trialkylammonium. Particularly preferably, Q represents sodium, lithium, trimethylsilyl, tetraethylammonium, tetra-n-butylammonium or tetramethylguanidinium, very particularly preferably sodium or lithium.

Azides of the formula (IV) are commercially available or can be prepared by known methods (cf. Houben-Weyl: Methoden der Organischen Chemie [Methods of Organic Chemistry], fourth edition, Organo-Stickstoff Verbindungen I [Organo-nitrogen compounds I], pages 1243-1290; Editor: D. Klamann).

Diluents suitable for carrying out the process (a) according to the invention are water, alcohols, ketones, nitrites or sulphoxides, or mixtures of these. Preference is given to using water/acetone mixtures, water/ethanol mixtures, DMSO or acetonitrile, particularly preferably water/acetone mixtures or water/ethanol mixtures.

A suitable catalyst is, for example, methyltrioctylammonium chloride (Aliquat 336) (cf. M. Es-Sayed, Phd thesis, University of Göttingen, 1992).

The reaction temperatures for carrying out the process (a) according to the invention can be varied within a relatively wide range. In general, the process is carried out at temperatures between 0° C. and 100° C., preferably between 30° C. and 70° C., particularly preferably between 40° C. and 60° C.

Some of the halides of the formula (m) required as starting materials for carrying out the process (a) according to the invention are known. Halides of the formula (III-b)

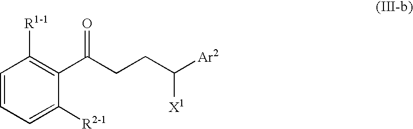

in which

X¹ represents chlorine, bromine or iodine and

R¹⁻¹, R²⁻¹ and Ar² are as defined above

are novel.

Halides of the formula (III-b) can be prepared by reacting

b) cyclopropanes of the formula (V-a)

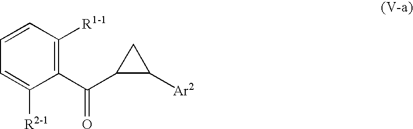

in which R¹⁻¹, R²⁻¹ and Ar² are as defined above

with a protic acid, if appropriate in the presence of a diluent.

Halides of the formula (III) can be prepared analogously. To this end, cyclopropanes of the formula (V)

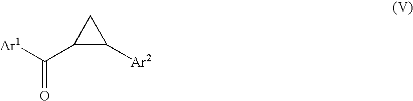

in which Ar¹ and Ar² are as defined above

are reacted according to process (b).

The formula (V-a) provides a general definition of the cyclopropanes required as starting materials for carrying out the process (b) according to the invention. In this formula, Ar² preferably, particularly preferably and very particularly preferably has those meanings which have already been mentioned in connection with the description of the starting materials of the formula (II) as being preferred, particularly preferred and very particularly preferred, respectively, for these radicals. R¹⁻¹ preferably represents fluorine or chlorine, R²⁻¹ preferably represents hydrogen, fluorine or chlorine.

Suitable protic acids for carrying out the process (b) according to the invention are hydrohalic acids. Preference is given to using HCl, HBr or HI, particularly preferably HCl.

Diluents suitable for carrying out the process (b) according to the invention are water or alcohols, preferably water.

The reaction temperatures for carrying out the process (b) according to the invention can be varied within a relatively wide range. In general, the process is carried out at temperatures between −20° C. and +60° C., preferably between 0° C. and 40° C., particularly preferably at room temperature.

Some of the cyclopropanes of the formula (V) required as starting materials for carrying out the process (b) according to the invention are known. Cyclopropanes of the formula (V-a)

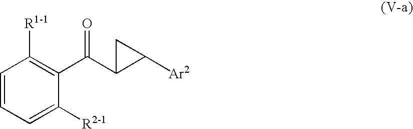

in which

R¹⁻¹, R²⁻¹ and Ar² are as defined above

are novel.

Cyclopropanes of the formula (V-a) can be prepared by reacting

c) chalcones of the formula (VI)

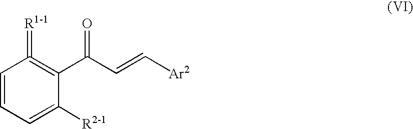

in which R¹⁻¹, R²⁻¹ and Ar² are as defined above

with a trialkylsulphoxonium ylide in the presence of a base and, if appropriate, in the presence of a diluent.

Cyclopropanes of the formula (V) can be prepared analogously.

The formula (VI) provides a general definition of the chalcones required as starting materials for carrying out the process (c) according to the invention. In this formula, Ar² preferably, particularly preferably and very particularly preferably has those meanings which have already been mentioned in connection with the description of the starting materials of the formula (II) as being preferred, particularly preferred and very particularly preferred, respectively, for these radicals. R¹⁻¹ preferably represents fluorine or chlorine, R²⁻¹ preferably represents hydrogen, fluorine or chlorine.

A trialkylsulphoxonium ylide which is preferably used for carrying out the process (c) according to the invention is trimethylsulphoxonium ylide.

Bases suitable for carrying out the process (c) according to the invention are alkali metal hydrides, alkali metal alkoxides and alkali metal hydroxides. Preference is given to using sodium hydride, potassium 2-methyl-2-propoxide, sodium methoxide or potassium hydroxide, particularly preferably sodium hydride.

Diluent suitable for carrying out the process (c) according to the invention are dimethyl sulphoxide, tetrahydroffuran, acetonitrile, toluene or diethylene glycol, and mixtures of these. Preference is given to using dimethyl sulphoxide (cf. Tetrahedron Asymmetry 1998, 9, 1035).

The reaction temperatures for carrying out the process (c) according to the invention can be varied within a relatively wide range. In general, the process is carried out at temperatures between −20° C. and +120° C., preferably between 0° C. and 60° C., particularly preferably between 20° C. and 40° C.

The chalcones of the formula (VI) required as starting materials for carrying out the process (c) according to the invention are known.

When carrying out the process according to the invention, it is possible to use all trialkylphosphines, triarylphosphines and trialkyl phosphites which can usually be used for this purpose (cf. Tetrahedron Lett. 1999, 40, 4825; Tetrahedron 1997, 53, 3693; Tetrahedron 1997, 55, 8353; J. Chem. Soc. Chem. Commun. 1982, 1224; Synthesis 1996, 123). Preference is given to using organophosphorus compounds, such as triphenylphosphine, tri-n-butylphosphine or trimethyl phosphite, particularly preferably triphenylphosphine.

It is furthermore possible to convert azides of the formula (II) by catalytic hydrogenation using, for example, the catalyst PtO₂, according to the process according to the invention into compounds of the formula (I) (cf J. Am. Chem. Soc. 1954, 76, 1231).

Further options for reducing azide compounds are described in the literature (cf. Houben-Weyl: Methoden der Organischen Chemie, fourth edition, Organo-Stickstoff Verbindungen II, pages 956-975; Editor: D. Klamann).

Diluents suitable for carrying out the process according to the invention are aliphatic or aromatic hydrocarbons, halogenated hydrocarbons or ethers. Preference is given to using pentane, hexane, heptane, benzene, toluene, tetrahydrofuran, diethyl ether, dioxane or acetonitrile, particularly preferably pentane, hexane or heptane.

The reaction temperatures for carrying out the process according to the invention can be varied within a relatively wide range. In general, the process is carried out at temperatures between −10° C. and +60° C., preferably between 0° C. and 40° C., particularly preferably at room temperature.

The process according to the invention and the processes (a), (b) and (c) are generally each carried out under atmospheric pressure. However, in each case it is also possible to operate under elevated or reduced pressure.

When carrying out the process according to the invention, in general 1 mol of trialkylphosphine and a suitable diluent are used per mole of azide of the formula (II). However, it is also possible to select other ratios of the reaction components. Work-up is carried out by customary methods. In general, the reaction mixture is concentrated in the presence of Florisil and then chromatographed using a mixture of n-hexane and ethyl acetate.

Some of the 2,5-bisaryl-Δ¹-pyrrolines of the formula (I) which can be prepared by the process according to the invention are known. Also known is their use for controlling pests. They are particularly suitable for controlling insects, arachnids and nematodes encountered in agriculture, in forests, in the protection of stored products and the protection of material and in the hygiene sector (see WO 00/21958, WO 99/59968, WO 99/59967 and WO 98/22438).

2,5-Bisaryl-Δ¹-pyrrolines of the formula (I-a)

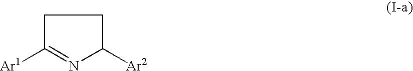

in which

Ar¹ is as defined above,

Ar² represents the radical

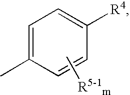

R⁴ and m are as defined above,

R⁵⁻¹ represents hydroxyl, trialkylsilyl, alkoxycarbonyl, —CONR⁷R⁸ or —NR⁷R⁸ and

R⁷ and R⁸ are as defined above

are novel.

Compounds of the formula (I-a) have very good insecticidal properties and can be used both in crop protection and in the protection of materials for controlling unwanted pests, such as insects. They are particularly suitable for controlling insects, arachnids and nematodes encountered in agriculture, in forests, in the protection of stored products and the protection of materials and in the hygiene sector (see WO 00/21958, WO 99/59968, WO 99/59967 and WO 98/22438).

The practice of the process according to the invention is illustrated by the examples below.

### PREPARATION EXAMPLE

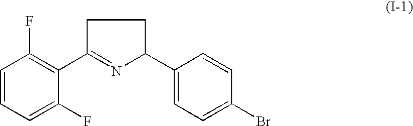

Step 1

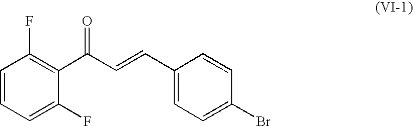

23.40 g of 2,6-difluoroacetophenone (0.15 mol), 27.75 g of 4-bromobenzaldehyde (0.15 mol), 60 ml of methanol and 150 ml of water are initially charged in a three-necked flask. At room temperature, 45 ml of aqueous sodium hydroxide solution (10% strength solution in water) are added dropwise, and the mixture is then stirred at room temperature overnight. The reaction mixture is cooled to 5° C. and the precipitate is filtered off and washed with 100 ml of cold methanol/water (1:3).

This gives 44.77 g (92% of theory) of (2E)-3-(4-bromophenyl)-1-(2,6-difluorophenyl)-2-propen-1-one of melting point 71° C.

HPLC: log P (pH 2.3)=3.98 (98% pure).

¹H-NMR spectrum (D₆-DMSO): δ=7.25-7.35 (3H, m), 7.54 (1H, d), 7.62-7.72 (3H, m), 7.76 (2H, d) ppm.

Step 2

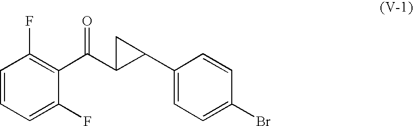

Under an atmosphere of argon, 4.50 g of sodium hydride (80% strength suspension in oil, 0.15 mol) are initially charged in 150 ml of DMSO. 33.0 g of trimethylsulphoxonium iodide (0.15 mol) are added a little at a time. After 2 hours of stirring at room temperature, a solution of 44.40 g of the compound (VI-1) (0.137 mol) in 200 ml of DMSO is added dropwise, and the mixture is stirred further at room temperature overnight. The reaction mixture is stirred into 2 litres of water and extracted twice with in each case 400 ml of ethyl acetate. The combined organic phases are washed once with 200 ml of water, dried over sodium sulphate, filtered and concentrated under reduced pressure. The residue is triturated with isopropanol and filtered off with suction.

This gives 32.36 g (64% of theory) of [2-(4-bromophenyl)cyclopropyl](2,6-difluorophenyl)methanone of melting point 64 to 65° C.

HPLC: log P (pH 2.3)=4.24 (97% pure).

¹H-NMR spectrum (CDCl₃): δ=1.57 (1H, m), 1.97 (1H, m), 2.59 (1H, m), 2.77 (1H, m), 6.95 (2H, m), 7.06 (2H, d), 7.40 (3H, m) ppm.

Step 3

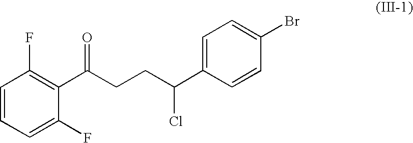

64.72 g (0.19 mol) of the compound (V-1) and 500 ml of concentrated hydrochloric acid (37% strength) are stirred at room temperature for 4 days. The reaction mixture is extracted twice with in each case 350 ml of dichloromethane. The combined organic phases are dried over sodium sulphate, filtered and concentrated under reduced pressure.

This gives 70.05 g (99% of theory) of 4-(4-bromophenyl)-4-chloro-1-(2,6-difluorophenyl)-1-butanone in the form of an oil which is reacted farther without purification.

HPLC: log P (pH 2.3)=4.52 (95% pure).

¹H-NMR spectrum (CD₃CN): δ=2.42 (2H, m), 3.01 (2H, m), 5.06 (1H, dd), 7.05 (2H, m), 7.37 (2H, d), 7.48-7.59 (3H, m) ppm.

Step 4

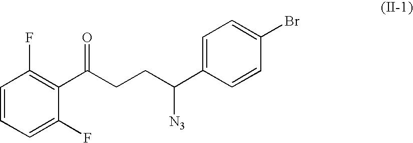

0.93 g (2.50 mmol) of the compound (III-1) is initially charged in 4 ml of acetone. At room temperature, a solution of 0.25 g of sodium azide (3.80 mmol) in 10 ml of water and 0.3 g of Aliquat 336 are added. The mixture is then stirred at 50° C. for 16 hours. The reaction mixture is cooled to room temperature, diluted with 20 ml of water and extracted three times with in each case 50 ml of ethyl acetate. The combined organic phases are dried over sodium sulphate, filtered and concentrated under reduced pressure.

This gives 1.12 g of 4-(4-bromophenyl)-4-azido-1-(2,6-difluorophenyl)-1-butanone as a crude product which is reacted further without purification.

HPLC: log P (pH 2.3)=4.47 (85% pure).

IR spectrum: \tilde{v}_{azide}=2900 cm⁻¹.

¹H-NMR spectrum (CD₃CN): δ=2.11 (2H, m), 2.94 (2H, m), 4.66 (1H, m), 7.05 (2H, m), 7.30 (2H, d), 7.50 (1H, m), 7.58 (2H, d) ppm.

Step 5

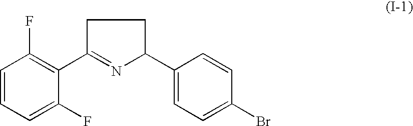

1.12 g of the crude product of (II-1) are initially charged in 100 ml of pentane. 0.65 g of triphenylphosphine (2.5 mmol) are added a little at a time, and the mixture is then stirred at room temperature overnight. 12 g of Florisil are added, and the mixture is evaporated to dryness under reduced pressure. The product is purified by flash chromatography (mobile phase: n-hexane/ethyl acetate 9:1).

This gives 0.51 g (59% of theory) (over steps 4 and 5) of 2-(4-bromophenyl)-5-(2,6-difluorophenyl)-3,4-dihydro-2H-pyrrole as an oil which crystallized over time (melting point 48° C.).

HPLC: log P (pH 2.3)=2.72 (100% pure).

¹H-NMR spectrum (CD₃CN): δ=1.76 (1H, m), 2.60 (1H, m), 3.03 (2H, m), 5.25 (1H, m), 7.10 (2H, m), 7.28 (2H, d), 7.45 (1H, m), 7.50 (2H, d) ppm.

Alternatively, steps 4 and 5 can be prepared using the following procedures.

Steps 4 and 5 (Alternative Route)

At room temperature, a solution of 70 g (0.187 mol) of 1-(2,6-difluorophenyl)-4-chloro-4-(4-bromophenyl)butan-1-one (III-1) is added to a solution of 19 g (0.29 mol) of sodium azide and 5 drops of Aliquat 336 in 100 ml of water, and the mixture is boiled under reflux for 10 hours. After cooling to room temperature, the oil that has separated off is dissolved in 1.61 of n-hexane, the aqueous phase is separated off and the n-hexane solution is dried with sodium sulphate. At room temperature, 49 g (0.187 mol) of triphenylphosphine are added a little at a time to this n-hexane solution of the resulting 1-(2,6-difluorophenyl)-4-azido-4-(4-bromophenyl)butan-1-one. With evolution of nitrogen, the temperature of the reaction mixture slowly increases to 31° C. The mixture is stirred at room temperature for 16 hours and the solvent is then distilled off under reduced pressure. This gives 70.4 g of an oily residue with contains about 48% of the title compound (in addition to triphenylphosphine and triphenylphosphine oxide) and is purified by silica gel chromatography (mobile phase: n-hexane/ethyl acetate 9:1-4:1).

This gives 34.6 g (35% of theory) of 2-(4-bromophenyl)-5-(2,6-difluorophenyl)-3,4-dihydro-2H-pyrrole.

HPLC: log P (pH 2.3)=2.71 (99.4% pure).

Compounds of the formulae (I), (II), (III), (V) and (VI) were prepared analogously to, the procedures above:

Pyrrolines of the Formula (I)

| | |
|---|---|
| | (I) |
| 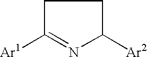 | |

| Ex. No. | Ar¹ | Ar² | logP* | m.p. |
|---|---|---|---|---|
| I-1 | 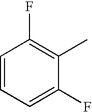 | 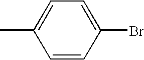 | 2.77 | 48° C. |
| I-2 | 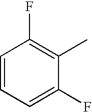 | 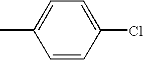 | 2.59 | |
| I-3 | 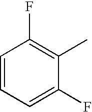 | 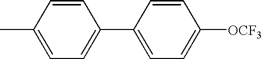 | 4.78 | 112° C. |
| I-4 | 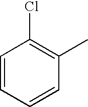 | 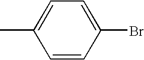 | 2.82 | |
| I-5 | 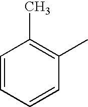 | 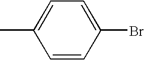 | 3.45 | |
| I-6 | 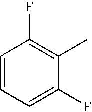 | 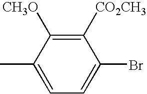 | 3.01 | 94° C. |
| I-7 | 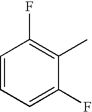 | 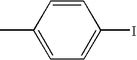 | 3.04 | 72-73° C. |
| I-8 | 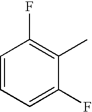 | 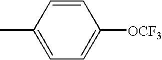 | 3.20 | |

Azides of the Formula (II)

| | |
|---|---|
| | (II) |
| 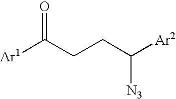 | |

| Ex. No. | Ar¹ | Ar² | logP* | ν_{azide} cm⁻¹ |
|---|---|---|---|---|
| II-1 | 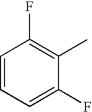 | 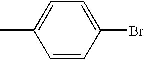 | 4.47 | 2900 |
| II-2 | 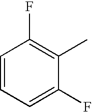 | 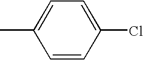 | | |
| II-3 | 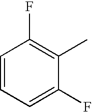 | 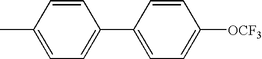 | | 2908 |
| II-4 | 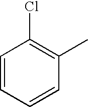 | 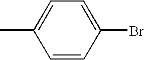 | | 2909 |
| II-5 | 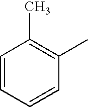 | 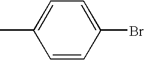 | | 2910 |
| II-6 | 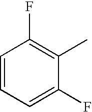 | 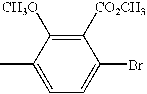 | 4.15 | 2905 |
| II-7 | 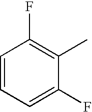 | 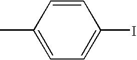 | 4.66 | 2905 |
| II-8 | 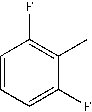 | 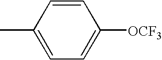 | 4.56 | 2910 |

Haloketones of the Formula (III)

| | |
|---|---|
| | (III) |
| 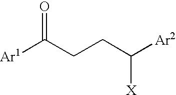 | |

| Ex. No. | Ar¹ | Ar² | X | logP* |
|---|---|---|---|---|
| III-1 | 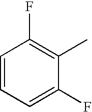 | 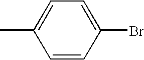 | Cl | 4.43 |
| III-2 | 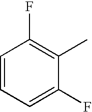 | 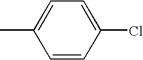 | Cl | |
| III-3 | 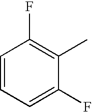 | 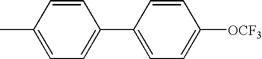 | Cl | 5.50 |
| III-4 | 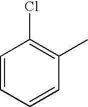 | 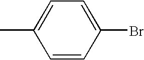 | Cl | 4.77 |
| III-5 | 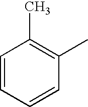 | 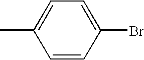 | Cl | 4.90 |
| III-6 | 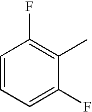 | 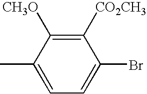 | Cl | 4.21 |
| III-7 | 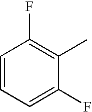 | 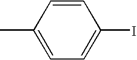 | Cl | 4.70 |
| III-8 | 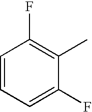 | 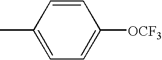 | Cl | 4.38 |
| III-9 | 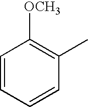 | 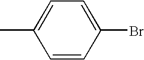 | Cl | 4.53 |
| III-10 | 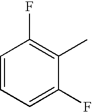 | 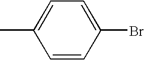 | Br | 4.61 |

Cyclopropanes of the Formula (V)

| | |
|---|---|
| | (V) |
| 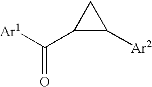 | |

| Ex. No. | Ar¹ | Ar² | logP* | m.p. |
|---|---|---|---|---|
| V-1 | 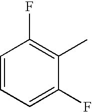 | 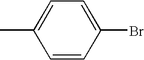 | 4.22 | |
| V-2 | 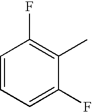 | 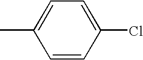 | | |
| V-3 | 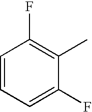 | 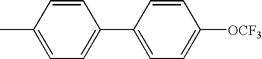 | 5.32 | 72° C. |
| V-4 | 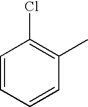 | 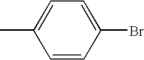 | 4.56 | |
| V-5 | 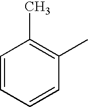 | 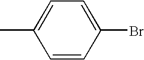 | 4.65 | |
| V-6 | 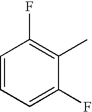 | 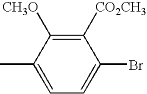 | 3.77 | |
| V-7 | 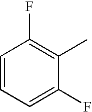 | 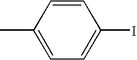 | 4.43 | |
| V-8 | 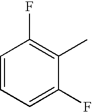 | 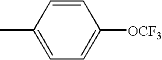 | 4.35 | |

Chalcones of the Formula (VI)

| | |
|---|---|
| | (VI) |
| 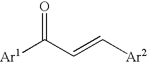 | |

| Ex. No. | Ar¹ | Ar² | logP* | m.p. |
|---|---|---|---|---|
| VI-1 | 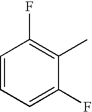 | 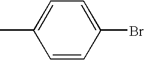 | 3.98 | 71° C. |
| VI-2 | 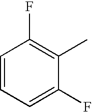 | 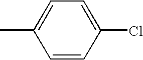 | | |
| VI-3 | 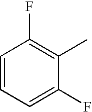 | 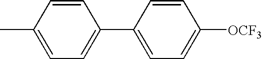 | 4.99 | |
| VI-4 | 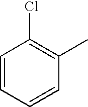 | 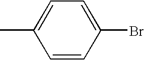 | 4.25 | |
| VI-5 | 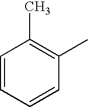 | 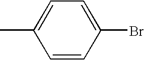 | 4.36 | |
| VI-6 | 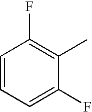 | 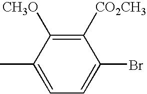 | 3.71 | |
| VI-7 | 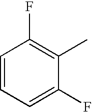 | 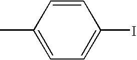 | 4.18 | |
| VI-8 | 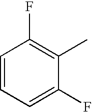 | 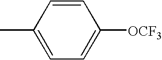 | 4.11 | |

The log P values given in the Tables and preparation examples above are determined in accordance with EEC Directive 79/831 Annex V.A8 by HPLC (High Performance Liquid Chromatography) on a reverse-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (of 3 to 16 carbon atoms) with known log P values (determination of the log P values by the retention times using linear interpolation between two successive alkanones).

The lambda max values were determined in the maxima of the chromatographic signals using the UV spectra from 200 nm to 400 nm.

1. Process for preparing 2,5-bisaryl-Δ¹-pyrrolines of the formula (I) 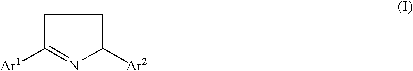
in which
Ar¹ represents the radical 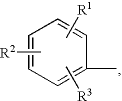
Ar² represents the radical 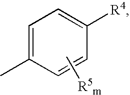
m represents 0, 1, 2.3 or 4,
R¹ represents halogen, cyano, nitro, alkyl, alkoxy, haloalkyl, haloalkoxy, alkoxyalkyl, —S(O)ₒR⁶ or —NR⁷R⁸,
R² and R³ independently of one another represent hydrogen, halogen, cyano, nitro, alkyl, alkoxy, haloalkyl, haloalkoxy, alkoxyalkyl, —S(O)ₒR⁶ or —NR⁷R⁸,
R⁴ represents halogen or one of the groupings below
(l) —X-A
(m) —B—Z-D
(n) —Y-E,
R⁵ represents halogen, hydroxyl, alkyl, alkoxy, haloalkyl, haloalkoxy, trialkylsilyl, alkoxycarbonyl, —CONR⁷R⁸, —S(O)ₒR⁶ or —NR⁷R⁸,
X represents a direct bond, oxygen, —S(O)ₒ—, NR₆—, carbonyl, carbonyloxy, oxycarbonyl, oxysulphonyl (OSO₂), alkylene, alkenylene, alkynylene, alkylenoxy, oxyalkylene, oxyalkylenoxy, —S(O)ₒ-alkylene, cyclopropylene or oxiranylene,
A represents phenyl, naphthyl or tetrahydronaphthyl, each of which is optionally mono- or polysubstituted by radicals from the list W¹, or represents 5- to 10-membered saturated or unsaturated heterocyclyl which contains one or more heteroatoms from the group consisting of nitrogen, oxygen and sulphur and is in each case optionally mono- or polysubstituted by radicals from the list W²,
B represents p-phenylene which is optionally mono- or disubstituted by radicals from the list W¹,
Z represents —(CH₂)ₙ, oxygen or —S(O)ₒ—,
D represents hydrogen, alkyl, alkenyl, alkynyl, haloalkyl, haloalkenyl, haloalkylsulphonyl or dialkylaminosulphonyl,
Y represents a direct bond, oxygen, sulphur, —SO₂—, carbonyl, carbonyloxy, oxycarbonyl, alkylene, alkenylene, alkynylene, haloalkylene, haloalkenylene, alkylenoxy, oxyalkylene, oxyalkylenoxy or thioalkylene,
E represents hydrogen, alkyl, alkenyl, alkynyl, haloalkyl, haloalkenyl, haloalkylsulphonyl or dialkylaminosulphonyl,
W¹ represents cyano, halogen, formyl, nitro, alkyl, trialkylsilyl, alkoxy, haloalkyl, haloalkenyl, haloalkoxy, haloalkenyloxy, alkylcarbonyl, alkoxycarbonyl, —S(O)ₒR⁶ or —SO₂NR⁷R⁸,
W² represents cyano, halogen, formyl, nitro, alkyl, trialkylsilyl, alkoxy, haloalkyl, haloalkoxy, haloalkenyloxy, alkylcarbonyl, alkoxycarbonyl or —S(O)ₙR⁶,
n represents 0, 1, 2, 3 or 4,
o represents 0, 1 or 2,
R⁶ represents hydrogen, alkyl or haloalkyl,
R⁷ and R⁸ independently of one another represent hydrogen, alkyl, haloalkyl, or together represent alkylene or alkoxyalkylene,
characterized in that
azides of the formula (II) 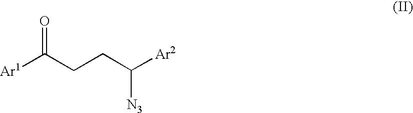
in which Ar¹ and Ar² are as defined above are reacted with a trialkylphosphine or a triarylphoshine or a trialkyl phosphite or a reducing agent in the presence of a diluent and, if appropriate, in the presence of a catalyst.

2. Process according to claim 1, characterized in that the starting materials used are azides of the formula (II) 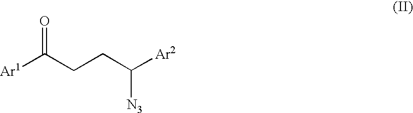
in which
Ar¹ represents the radical 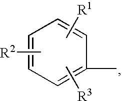
Ar² represents the radical 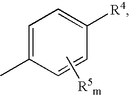
m represents 0, 1, 2 or 3,
R¹ represents halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸,
R² and R³ independently of one another represent hydrogen, halogen, cyano, nitro, C₁-C₆-alkyl, C₁-C₆-alkoxy, C₁-C₆-haloalkyl, C₁-C₆-haloalkoxy, C₁-C₆-alkoxy-C₁-C₆-alkyl, —S(O)ₒR⁶ or —NR⁷R⁸,
R⁴ represents fluorine, chlorine, bromine, iodine or one of the groupings below
(l) —X-A
(m) —B—Z-D
(n) —Y-E,
R⁵ represents halogen, hydroxyl, C₁-C₆-alkyl, C₁-C₆-alkoxy, C₁-C₆-haloalkyl, C₁-C₆-haloalkoxy, tri(C₁-C₆-alkyl)silyl, C₁-C₆-alkoxycarbonyl, —CONR⁷R⁸, —S(O)ₒR⁶ or —NR⁷R⁸,
X represents a direct bond, oxygen, —S(O)ₙ—, NR₆—, carbonyl, carbonyloxy, oxycarbonyl, oxysulphonyl (OSO₂), C₁-C₄-alkylene, C₂C₄-alkenylene, C₂-C₄-alkynylene, C₁-C₄-alkylenoxy, C₁-C₄-oxyalkylene, C₁-C₄-oxyalkylenoxy, —S(O)ₒ-C₁-C₄-alkylene, cyclopropylene or oxiranylene,
A represents phenyl, naphthyl or tetrahydronaphthyl, each of which is optionally mono- to tetrasubstituted by radicals from the list W¹, or represents 5- to 10-membered heterocyclyl which contains 1 or 2 aromatic rings and 1 to 4 heteroatoms, selected from a combination of 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl), and is in each case optionally mono- to tetrasubstituted by radicals from the list W²,
B represents p-phenylene which is optionally mono- or disubstituted by radicals from the list W¹,
Z represents —(CH₂)ₙ—, oxygen or —S(O)ₒ—,
D represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl) aminosulphonyl,
Y represents a direct bond, oxygen, sulphur, —SO₂—, carbonyl, carbonyloxy, oxycarbonyl, C₁-C₆-alkylene, C₂-C₆-alkenylene, C₂-C₆alkynylene, C₁-C₆-haloalkylene, C₂-C₆-haloalkenylene, C₁-C₄-alkylenoxy, C₁-C₄-oxyalkylene, C₁-C₄-oxyalkylenoxy or C₁-C₄-thioalkylene,
E represents hydrogen, C₁-C₆-alkyl, C₂-C₆-alkenyl, C₂-C₆-alkynyl, C₁-C₆-haloalkyl, C₂-C₆-haloalkenyl, C₁-C₆-haloalkylsulphonyl or di(C₁-C₆-alkyl)aminosulphonyl,
W¹ represents cyano, halogen, formyl, nitro, C₁-C₆-alkyl, tri(C₁-C₄alkyl)silyl, C₁-C₆-alkoxy, C₁-C₆-haloalkyl, C₂-C₆-haloalkenyl, C₁-C₆-haloalkoxy, C₂-C₆-haloalkenyloxy, C₁-C₆-alkylcarbonyl, C₁-C₆alkoxycarbonyl, —S(O)ₒR⁶ or —SO₂NR⁷R⁸,
W² represents cyano, halogen, formyl, nitro, C₁-C₆-alkyl, tri(C₁-C₄alkyl)silyl, C₁-C₆-alkoxy, C₁-C₆-haloalkyl, C₁-C₆-haloalkoxy, C₂-C₆-haloalkenyloxy, C₁-C₆-alkylcarbonyl, C₁-C₆-alkoxycarbonyl or —S(O)ₒR⁶,
n represents 0, 1, 2, 3 or 4,
o represents 0, 1 or 2,
R⁶ represents hydrogen, C₁-C₆-alkyl or C₁-C₆-haloalkyl,
R⁷ and R⁸ independently of one another represent hydrogen, C₁-C₆-alkyl, C₁-C₆-haloalkyl, or together represent C₂-C₆-alkylene or C₁-C₄-alkoxy-C₁-C₄-alkylene (for example morpholine).

3. Process according to claim 1, characterized in that the starting materials used are azides of the formula (II) 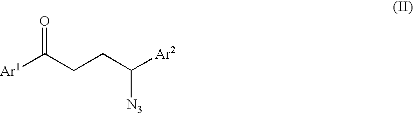
in which
Ar¹ represents the radical 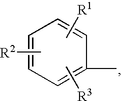
Ar² represents the radical 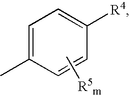
m represents 0, 1 or 2,
R¹ represents fluorine, chlorine, bromine, C₁-C₆-alkyl, C₁-C₆-alkoxy, in each case fluorine- or chlorine-substituted C₁-C₆-alkyl or C₁-C₆alkoxy,
R² and R³ independently of one another represent hydrogen, fluorine, chlorine, bromine, iodine, C₁-C₆-alkyl, C₁-C₆-alkoxy, in each case fluorine- or chlorine-substituted C₁-C₆-alkyl or C₁-C₆-alkoxy,
R⁴ represents chlorine, bromine, iodine or one of the groupings below
(l) —X-A
(m) —B—Z-D
(n) —Y-E,
R⁵ represents fluorine, chlorine, bromine, iodine, hydroxyl, C₁-C₆-alkyl, C₁-C₆-alkoxy, in each case fluorine- or chlorine-substituted C₁-C₆alkyl or C₁-C₆-alkoxy, C₁-C₄-alkoxycarbonyl, —CONR⁷R⁸, —S(O)ₙR⁶ or —NR⁷R⁸,
X represents a direct bond, oxygen, sulphur, —SO₂—, carbonyl, carbonyloxy, oxycarbonyl, oxysulphonyl (OSO₂), C₁-C₄-alkylene, C₂C₄-alkenylene, C₂-C₄-alkynylene, C₁-C₄-alkylenoxy, C₁-C₄-oxyalkylene, C₁-C₄-oxyalkylenoxy, —S(O)ₙ—C₁-C₄-alkylene, cyclopropylene or oxiranylene,
A represents phenyl, naphthyl or tetrahydronaphthyl, each of which is optionally mono- to trisubstituted by radicals from the list W¹, or represents 5- to 10-membered heterocyclyl which contains 1 or 2 aromatic rings and 1 to 4 heteroatoms, selected from a combination of 0 to 4 nitrogen atoms, 0 to 2 oxygen atoms and 0 to 2 sulphur atoms (in particular tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, quinolinyl or isoquinolinyl), and is in each case optionally mono- to trisubstituted by radicals from the list W²,
B represents p-phenylene which is optionally mono- or disubstituted by radicals from the list W¹,
Z represents —(CH₂)ₙ—, oxygen or —S(O)ₒ—,
D represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₄-alkylsulphonyl; or represents di(C₁-C₄alkyl)aminosulphonyl,
Y represents a direct bond, oxygen, sulphur, —SO₂—, carbonyl, carbonyloxy, oxycarbonyl, C₁-C₆-alkylene, C₂-C₆-alkenylene, C₂-C₆alkynylene; in each case fluorine- or chlorine-substituted C₁-C₆-alkylene or C₂-C₆-alkenylene; represents C₁-C₄-alkylenoxy, C₁-C₄-oxyalkylene, C₁-C₄-oxyalkylenoxy or C₁-C₄-thioalkylene,
E represents hydrogen, C₁-C₆-alkyl, C₂-C₆-alkenyl, C₂-C₆-alkynyl; in each case fluorine- or chlorine-substituted C₁-C₆-alkyl, C₂-C₆-alkenyl or C₁-C₆-alkylsulphonyl; or represents di(C₁-C₆alkyl)aminosulphonyl,
W¹ represents cyano, fluorine, chlorine, bromine, iodine, formyl, nitro, C₁-C₄-alkyl, C₁-C₄-alkoxy; in each case fluorine- or chlorine-substituted C₁-C₄-alkyl, C₂-C₄-alkenyl, C₁-C₄-alkoxy or C₂-C₆alkenyloxy; or represents C₁-C₄-alkylcarbonyl, C₁-C₄-alkoxycarbonyl, —S(O)ₒR⁶ or —SO₂NR⁷R⁸,
W² represents cyano, fluorine, chlorine, bromine, formyl, nitro, C₁-C₄alkyl, C₁-C₄-alkoxy; in each case fluorine- or chlorine-substituted C₁-C₄-alkyl, C₁-C₄-alkoxy or C₂-C₆-alkenyloxy, or represents C₁-C₄alkylcarbonyl, C₁-C₄-alkoxycarbonyl, —S(O)ₒR⁶,
n represents 0, 1, 2 or 3,
o represents 0, 1 or 2,
R⁶ represents C₁-C₆-alkyl or in each case fluorine- or chlorine-substituted methyl or ethyl,
R⁷ and R⁸ independently of one another represent C₁-C₆-alkyl, in each case fluorine- or chlorine-substituted C₁-C₆-alkyl, or together represent C₄C₅-alkylene or represent —(CH₂)₂—O—(CH₂)₂—.

4. Process according to claim 1, characterized in that the starting materials used are azides of the formula (II) 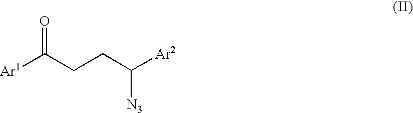
in which
Ar¹ represents the radical 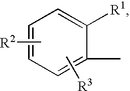
Ar² represents the radical 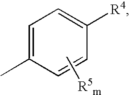
m represents 0, 1 or 2,
R¹ represents fluorine, chlorine, bromine, methyl or methoxy,
R² and R³ independently of one another represent hydrogen, fluorine, chlorine, bromine, methyl or methoxy,
R⁴ represents chlorine, bromine or one of the groupings below
(l) —X-A
(m) —B—Z-D
(n) —Y-E,
R⁵ represents fluorine, chlorine, bromine, hydroxyl, methyl, ethyl, methoxy, ethoxy, trifluoromethyl, difluoromethoxy, trifluoromethoxy, —CO₂CH₃ or —SO₂CF₃,
X represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S(O)ₒ—CH₂— or —S(O)ₙ—(CH₂)₂—,
A represents phenyl which is optionally mono- or disubstituted by radicals from the list W¹, or represents tetrazolyl, furyl, benzofuryl, thienyl, benzothienyl, pyrrolyl, indolyl, oxazolyl, benzoxazolyl, isoxazyl, imidazyl, pyrazyl, thiazolyl, benzothiazolyl, pyridyl, pyrimidinyl, pyridazyl, triazinyl, triazyl, each of which is optionally mono- or disubstituted by radicals from the list W²,
B represents p-phenylene which is optionally monosubstituted by radicals from the list W¹,
Z represents oxygen, sulphur or —SO₂—,
D represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂,
Y represents a direct bond, oxygen, sulphur, —SO₂—, carbonyl, —CH₂—, —(CH₂)₂—, —CH═CH— (E or Z), —C≡C—, —CH₂O—, —(CH₂)₂O—, —OCH₂—, —OCH₂O—, —O(CH₂)₂O—, —S—CH₂— or —S(CH₂)₂—,
E represents hydrogen, methyl, ethyl, n-propyl, isopropyl, n-butyl, isobutyl, sec-butyl, tert-butyl, 2-propenyl, butenyl, propargyl, butynyl, —CF₃, —CHF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —SO₂CF₃, —SO₂(CF₂)₃CF₃ or —SO₂NMe₂,
W¹ represents cyano, fluorine, chlorine, bromine, formyl, methyl, n-butyl, isobutyl, sec-butyl, tert-butyl, methoxy, ethoxy, n-propoxy, n-butoxy, isobutoxy, sec-butoxy, tert-butoxy, trifluoromethoxy, difluoromethoxy, —CF₃, —CBF₂, —CClF₂, —CF₂CHFCl, —CF₂CH₂F, —CF₂CCl₃, —CH₂CF₃, —CF₂CHFCF₃, —CH₂CF₂H, —CH₂CF₂CF₃, —CF₂CF₂H, —CF₂CHFCF₃, —OCH₂CF₃, —SCF₃, —SCHF₂, —SOCHF₂, —SO₂CHF₂, —SOCF₃, —SO₂CF₃ or —SO₂NMe₂,
W² represents fluorine, chlorine, bromine, methyl, isopropoxy, tert-butoxy, trifluoromethyl, trifluoromethoxy, difluoromethoxy, trifluoromethylthio, —CO₂CH₃ or —SO₂CF₃,
o represents 0, 1 or 2.

5. Process according to claim 1, characterized in that the starting material used is the compound of the formula (II-1)

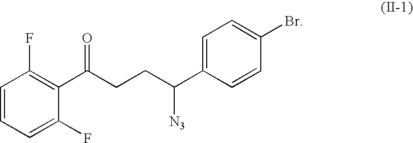

6. Process according to claim 1, characterized in that the starting material used is the compound of the formula (II-2) (II-2).

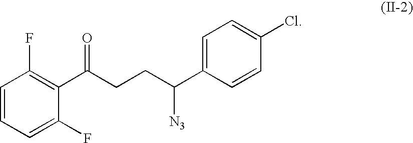

7. Process according to claim 1, characterized in that the starting material used is the compound of the formula (II-3)

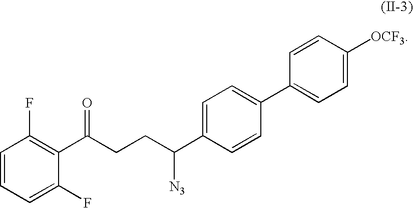

8. Process according to claim 1, characterized in that the reaction is carried out in the presence of triphenylphosphine, tri-n-butylphosphine or trimethyl phosphite.

9. Process according to claim 8, characterized in that the reaction is carried out in the presence of triphenylphosphine.

10. Process according to claim 1, characterized in that azides of the formula (II) are converted by catalytic hydrogenation into compounds of the formula (I).

11. Process according to claim 10, characterized in that azides of the formula (II) are hydrogenated in the presence of the catalyst PtO₂.

12. Process according to claim 1, characterized in that the reaction is carried out in the presence of a diluent from the group of the aliphatic or aromatic hydrocarbons, the halogenated hydrocarbons or their ethers.

13. Process according to claim 12, characterized in that the reaction is carried out in the presence of a diluent from the group consisting of pentane, hexane, heptane, benzene, toluene, tetrahydrofuran, diethyl ether, dioxane and acetonitrile.

14. Process according to claim 1, characterized in that the reaction is carried out at temperatures between −10° C. and +60° C.

15. Compounds of the formula (II-a) 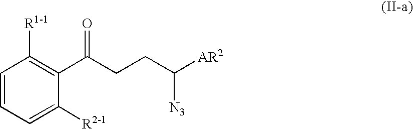
in which
R¹⁻¹ represents fluorine or chlorine,
R²⁻¹ represents hydrogen, chlorine or fluorine and
Ar² is as defined in any of claims 1 to 4.

16. Compounds of the formula (III-b) 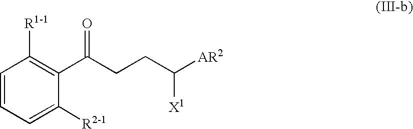
in which
R¹⁻¹ represents fluorine or chlorine,
R²-1 represents hydrogen, chlorine or fluorine,
X¹ represents chlorine, bromine or iodine and
Ar² is as defined in any of claims 1 to 4.

17. Compounds of the formula (V-a) 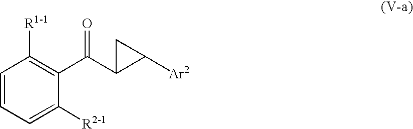
in which
R¹⁻¹ represents fluorine or chlorine,
R²⁻¹ represents hydrogen, chlorine or fluorine and
Ar² is as defined in any of claims 1 to 4.

18. 2,5-Bisaryl-Δ¹-pyrrolines of the formula (I-a) 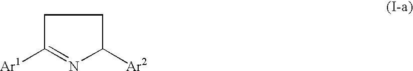
in which
Ar¹ is as defined in any of claims 1 to 4,
Ar² represents the radical 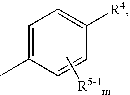
R⁴ and m are as defined in any of claims 1 to 4,
R⁵⁻¹ represents hydroxyl, trialkylsilyl, alkoxycarbonyl, —CONR⁷R⁸ or —NR⁷R⁸ and
R⁷ and R⁸ are as defined in any of claims 1 to 4.
